(19) United States
(12) Patent Application Publication
(10) Pub. No.: US 2013/0035590 A1
(43) Pub. Date: Feb. 7, 2013

(54) SYSTEMS AND METHODS TO ASSIST WITH INTERNAL POSITIONING OF INSTRUMENTS

(71) Applicant: SONOSITE, INC., Bothell, WA (US)

(72) Inventors: Qinglin Ma, Woodinville, WA (US); Paul Dunham, Bothell, WA (US); Nikolaos Pagoulatos, Bothell, WA (US); James M. Gilmore, Bothell, WA (US); Lee D. Dunbar, Bothell, WA (US); Kyle S. Johnston, Sammamish, WA (US)

(73) Assignee: SONOSITE, INC., Bothell, WA (US)

(21) Appl. No.: 13/648,244

(22) Filed: Oct. 9, 2012

Related U.S. Application Data
(62) Division of application No. 12/752,595, filed on Apr. 1, 2010, now abandoned.

(51) Int. Cl. A61B 8/13

(52) U.S. Cl. 600/424

(57) ABSTRACT

Systems and methods which facilitate the correct placement of an instrument internal to an object aided by an overlay superimposed on an image are disclosed. Exemplary embodiments facilitate placement of a needle tip within a patient's body using on overlay superimposed on a sonographic image. A superimposed overlay of embodiments is created by monitoring a fixed point of an external portion of the instrument in relation to an imaging transducer. Superimposed overlays provided according to embodiments provide one or more graphical target designator and one or more graphical instrument designator which, when controlled to be disposed in a predetermined position, indicate proper placement of the instrument.

### TECHNICAL FIELD

This disclosure relates to systems and methods for aiding interventional procedures, and more particularly to systems and methods for assisting internal positioning of instruments using optical positioning in combination with imaging.

### BACKGROUND OF THE INVENTION

Many medical procedures require precise positioning of an instrument internal to a patient. For example, interventional instruments, such as needles or catheters, are used to deliver medication or other fluids directly into an artery or vein or near a nerve within or internal to a patient's body. it is now common practice to use real-time ultrasound imaging to aid in the proper placement of the instrument.

The ultrasound imaging most often used provides a two-dimensional image plane. There are two commonly used methods to use real-time ultrasound imaging to aid in the placement of an instrument: the in-plane method wherein the instrument trajectory is in the ultrasound image plane; and the out-of-plane method wherein the instrument trajectory is out of the ultrasound image plane. That is, in such procedures the ultrasound transducer can be positioned along either the longitudinal axis of the instrument, often referred to as an in-plane technique (referring to the instrument being disposed longitudinally in the image plane of the ultrasound transducer), or transverse thereto, often referred to as an out-of-plane technique (referring to the instrument being disposed transverse or orthogonal to the image plane of the ultrasound transducer).

For the foregoing instrument placement applications it is often thought best to have both the target and the instrument in the imaging plane (in-plane method) for planning the trajectory. For example, in anesthesia and MSK applications the in-plane method is preferred because it can provide better visualization of the needle by being able to view the shaft of the needle. However, pickline and central venous catheter (CVC) applications most often use the out-of-plane method in order to view both the carotid artery and jugular vein simultaneously to avoid puncturing the artery.

At least two major difficulties exist for a practitioner using ultrasound image guided procedures. One such difficulty is the inability to know where the tip of the needle is for either the in-plane or out-of-plane methods. Another such difficulty is the hand-eye coordination demanded to keep the needle inside the thin imaging plane for the in-plane method. Furthermore, breathing, heart beat, and other movement can cause a change of relative position of the needle and the transducer, which is out of the control of the patient and physician.

Instruments may be positioned free-hand, without the use of positioning devices or guides, and thus not be precisely in either an in-plane or out-of-plane orientation. In the free-hand situation, it is often very difficult to know where the tip of a needle is located. Thus, techniques such as watching for tissue movement or watching the reaction after injecting a small amount of fluid are used to infer where the tip is located. Such methods used to infer instrument locations are therefore unreliable and cumbersome.

Various needle guides or biopsy guides have been developed to try to keep the needle inside the imaging plane, or predict the depth where the needle is going to insect the imaging plane for the out-of-plane approach. For example, a needle guide may be affixed to an ultrasound transducer to control the trajectory of the needle such that the portion of the needle inserted into a patient is guided within the image plane (in-plane method) or to intersect the image plane at a predetermined depth (out-of-plane method). However, such needle guides cannot provide the user with information regarding where the tip is in real-time.

Additionally, various spatial location systems have been tried to detect and track where the needle tip is. For example, position sensors such as electromagnetic sensors that are mounted on both the needle and the transducer are the most often used method for implementing a spatial location system. Although the use of such electromagnetic sensors have been shown to provide detection and tracking of a needle tip during some procedures, such spatial location systems are cumbersome, expensive and have the potential to interfere with bio-medical devices (e.g., patient pacemakers) and instruments (e.g., bio-telemetry) which are near where the procedure is being performed.

A gyronieter or potentiometer placed on a probe has also been tried for the out-of-plane method to provide information to a user. This technique predicts where the intersection point on the imaging plane is if the angle of insertion is changed. However, it does not provide any information regarding where the tip is located.

Another attempt to provide guidance for needle placement has been to use a laser beam on the needle to provide a visual guide to help align the needle with the imaging plane for an in-plan method. However, such laser beam implementations assume that some external markings on the transducer are aligned with the imaging plane and it requires the user looking down and to the side on the transducer. Once the user looks up to the image display, most often the relative position of the needle and transducer is changed. Therefore, this technique is not too practical and effective in practice.

U.S. Pat. No. 7,244,234 describes a guidance system using a transducer that has an array of Hall effect sensors built-in and a magnet mounted on the instrument. This technique suffers from the disadvantages described above with respect to other techniques which use electromagnetic sensors. Moreover, this technique requires significant modification of the existing conventional ultrasound tranducer configuration and housing design to accommodate a sterilizable seal. Furthermore, due to its requirement of proximity of the Hall effect sensors and the magnet, this technique is not very practical for use in an out-of-plane method.

When an out-of-plane technique is used, the ultrasound transducer is often utilized to image the desired target. Thus, as the instrument (e.g., needle) is being positioned, the clinician will only see the image of the cross section of the tip of the instrument, which is a small dot, as the tip enters the imaging plane. The clinician will not be able to determine where the tip is after it passes the imaging plane. When an in-plane technique is used, the ultrasound transducer is typically utilized to image both the target and the shaft of the instrument. Thus, the image will show the progress of the instrument, but will not necessarily able to display or clearly display the tip of the instrument sue to hand-eye coordination issues (e.g., the needle is generally not perfectly located in the imaging plane). Nevertheless, the clinician can employ alternative techniques to identify the instrument within the image. For example, the clinician can jiggle the instrument to cause tissue or other internal structure to move, whereby this movement can be seen in the resulting image. Inferences can be drawn from the visible movement by the clinician as to where the tip of the instrument is presently located, Another method for determining where the tip of the instrument is presently located is to inject a small amount of fluid and observe visible changes within the resulting image. However, both methods cannot pinpoint where the tip of the needle is, but rather can only give a proximity.

From the above, it can be appreciated that when using the techniques discussed above the clinician must often guess where the tip of the instrument is and, based on this “best guess” estimation, perform the desired procedure. However, various tissue such as veins, arteries, and nerves are often disposed in close proximity and thus it is important to be able to precisely identify where the tip of the instrument is during the procedure in real-time so that procedures (such as medicine delivery, wire insertion, etc.) are not performed with respect to an unintended target or otherwise to be more effective.

### BRIEF SUMMARY OF THE INVENTION

The present invention is directed to systems and methods which facilitate more precise placement of an instrument (such as a needle, catheter, stent, endoscope, angioplasty balloon, etc.) internal to an object, such as within the body of a patient, aided by an overlay superimposed on an image, such as a real-time ultrasound image. A superimposed overlay of embodiments is created by monitoring a fixed point of an external portion of the instrument in relation to an imaging transducer (e.g., ultrasound transducer). Superimposed overlays provided according to embodiments of the invention provide one or more predicted intersection pip or other graphical target designator and one or more instrument pip or other graphical instrument designator which, when controlled to be disposed in a predetermined position (e.g., concentrically overlapping), indicate proper placement of the instrument.

The foregoing target and instrument pips may be utilized to graphically represent any desired portion of a target structure or instrument, For example, a predicted intersection pip may represent a tissue lumen and an instrument pip may represent the tip of a needle instrument.

In embodiments of the invention, a fixed external point of the instrument is referenced to an imaging transducer by light (e.g., laser, light emitting diode (LED), infrared, etc.) passing between these two components. For example, a light transmitter (e.g., laser source) may be disposed upon either the external portion of the instrument or the imaging transducer and a light receiver (e.g., photosensitive array) may be correspondingly disposed upon the other of the imaging transducer and the external portion of the instrument for passing light between these two components. The light as detected by the foregoing light receiver is preferably used to reference the position of the instrument relative to the imaging transducer.

Multiple transmitter and receivers may also be used to obtain the relative location of a predetermined portion of an instrument, such as through the use of triangulation. For example, multiple light transmitters may be disposed upon either the external portion of the instrument or the imaging transducer and/or multiple light receivers may be disposed upon the other of the imaging transducer and the external portion of the instrument. Triangulation techniques may be utilized with the light as detected by the light receiver(s) to provide information regarding the orientation and position of the instrument relative to the imaging transducer.

An instrument guide, such as a needle guide, may be utilized to provide control of instrument movement, and thus provide information with respect to the orientation of the instrument (e.g., to determine the plane of instrument insertion) with respect to the imaging transducer. In situations where an instrument guide is not used, triangulation techniques may be used to provide information with respect to the orientation of the instrument (e.g., to determine the plane of instrument insertion) with respect to the imaging transducer.

Embodiments of the invention utilize available information regarding the orientation, position, and/or movement of an instrument relative to an imaging transducer to determine where a portion of the instrument of interest (e.g., the tip) is in relation to a target. For example, by knowing both the angle of attack of the instrument with respect to the transducer and the structural dimensions of the instrument, embodiments of the invention operate to calculate the position at any time of any desired portion of the instrument (e.g., the instrument tip). The calculated position of such a desired portion of the instrument within the object may then be superimposed (e.g., using an instrument pip and predicted intersection pip) onto an image generated using the imaging transducer, thereby allowing a clinician or other user to visualize the placement of the instrument.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. it should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWING

For a more complete understanding of the present invention, reference is now made to the following descriptions taken in conjunction with the accompanying drawing, in which:

FIG. 1a shows an illustration of an embodiment of the invention adapted to facilitate positioning an instrument using an out-of-plane technique;

FIG. 1b shows a superimposed overlay, including an instrument pip and predicted intersection pip, on an image according to an embodiment of the invention;

FIG. 1c shows an illustration of the embodiment of FIG. 1a wherein the instrument tip is positioned in the image plane of the imaging transducer;

FIG. 1d shows a superimposed overlay, including an instrument pip and predicted intersection pip, on an image corresponding to the instrument position shown in FIG. 1c according to an embodiment of the invention;

FIG. 1e shows an illustration of the embodiment of FIG. 1a Wherein the instrument tip has traversed the image plane of the imaging transducer;

FIG. 1f shows a superimposed overlay, including an instrument pip and predicted intersection pip, on an image corresponding to the instrument position shown in FIG. 1e according to an embodiment of the invention;

FIG. 2a shows a schematic view of a system adapted according to embodiments of the invention;

FIGS. 2h-2d illustrate operation of the embodiment of FIG. 2a to provide location determinations for an instrument;

FIGS. 3a-3c show geometric relationships for calculating instrument positioning according to embodiments of the invention;

FIGS. 4a and 4b illustrate a calibration procedure and use of an optical sensor for computation of the instrument tip coordinates with respect to an image plane according to an embodiment of the invention;

FIG. 5 shows detail with respect to the distribution of functional blocks of an imaging system adapted according to embodiments of the invention;

FIG. 6a shows an illustration of an embodiment of the invention.

adapted to facilitate positioning an instrument using an in-plane technique;

FIG. 6b shows a superimposed overlay, including a graphical instrument designator, on an image corresponding to the predicted instrument path trajectory and tip position shown in FIG. 6a according to an embodiment of the invention;

FIG. 7a shows an embodiment of the invention adapted to facilitate the detection of the relative position of the instrument with respect to the imaging plane;

FIGS. 7b and 7c show a graphical representation of the relative position of an instrument plane to a imaging plane according to embodiments of the invention; and

FIG. 7d shows a graphic display corresponding to the embodiment of FIG. 5a.

### DETAILED DESCRIPTION OF THE INVENTION

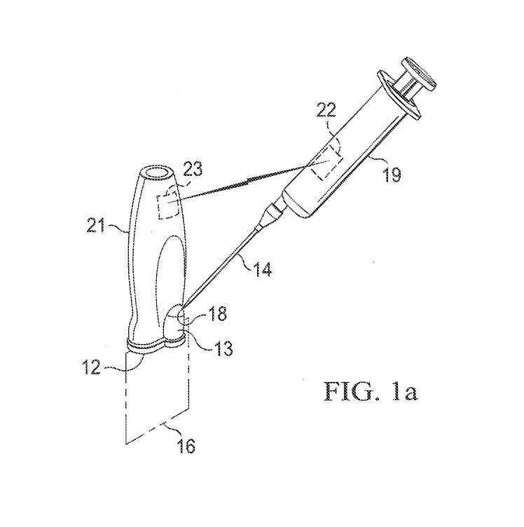

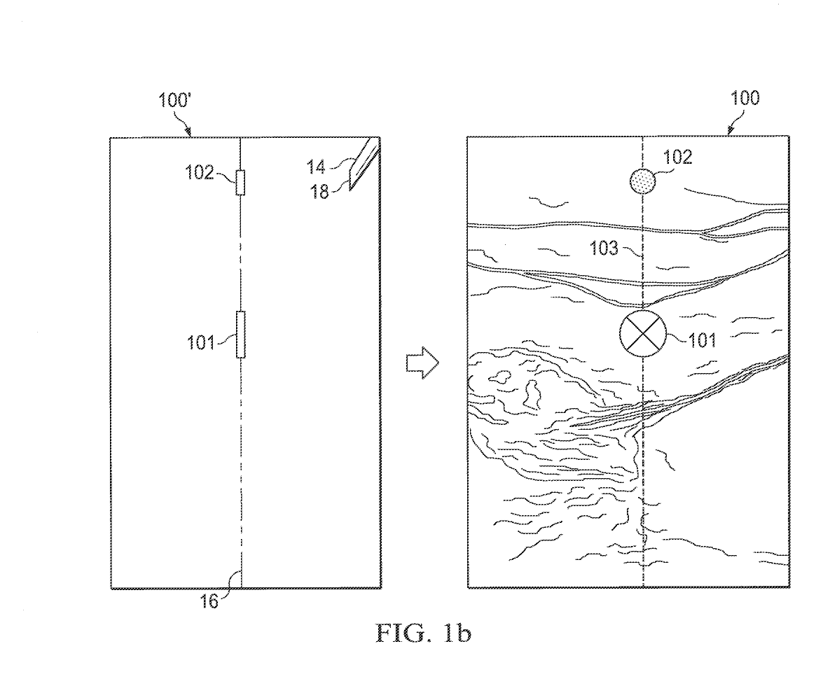

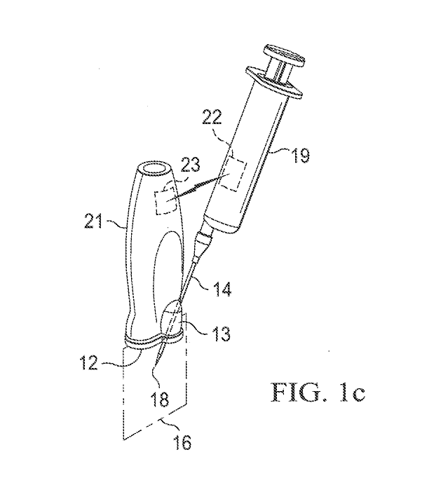

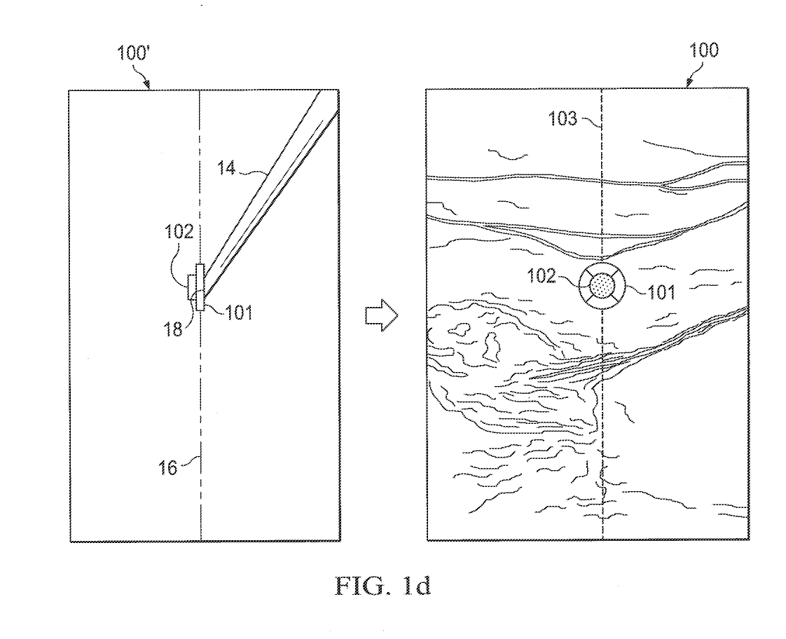

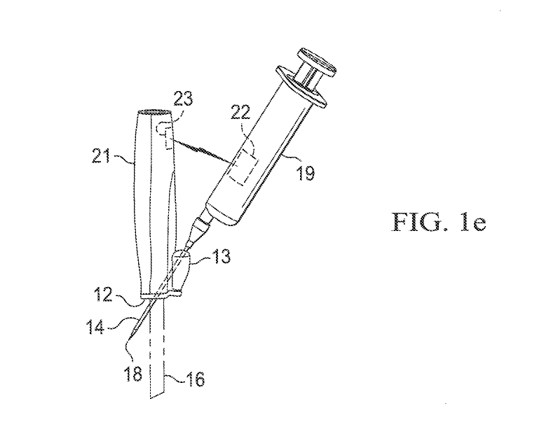

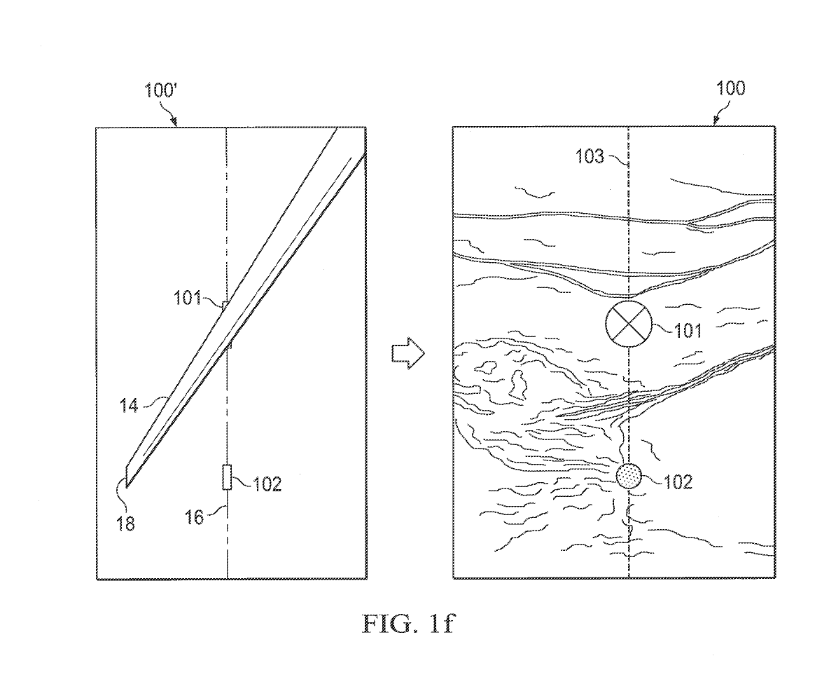

FIG. 1a shows an illustration of an embodiment of the invention adapted to facilitate positioning an instrument using an out-of-plane technique. Imaging transducer 21, such as may comprise an ultrasound transducer or other imaging transducer Configuration, obtains imaging information from an imaging area or volume, shown here as image plane 16, within an object (not shown). The object being imaged may comprise a portion of a human body, for example. In operation, imaging transducer 21, typically operable in combination with a host system unit such as may comprise an ultrasound system unit or other appropriate system unit, is used to provide an image of features of the object beneath surface 12 which would otherwise be invisible to the naked eye. Detail with respect to imaging systems which may be adapted according to the concepts of the present invention is provided in co-pending and commonly assigned U.S. patent application Ser. No. 12/467,899 entitled “Modular Apparatus for Diagnostic Ultrasound,” the disclosure of which is hereby incorporated herein by reference.

Imaging transducer 21 may be utilized to generate an image to facilitate positioning of instrument 14 (e.g., a biopsy needle or other instrument) within the object, such as to dispose tip 18 at or in a desired target. In the illustration of FIG. 1a, tip 18 of instrument 14 is positioned in front of imaging transducer 21 for insertion into the object being imaged. Imaging transducer 21 of the illustrated embodiment is shown fitted with needle guide 13 operable to provide at least some control of movement of instrument 14, and thus provide information with respect to the orientation of the instrument with respect to imaging transducer 21. A needle guide such as shown in co-pending and commonly assigned U.S. patent application Ser. No. 12/499,908 entitled “Device for Assisting the Positioning of Medical Devices,” the disclosure of which is hereby incorporated herein by reference, may be used to provide relative positioning of instrument 14 and imaging transducer 21 according to embodiments of the invention.

Instrument 14 is shown with portion 19 which remains external to the object during a desired procedure. Portion 19 of embodiments can be, for example, a syringe, the head of the instrument, or any portion beyond the portion of the instrument to be disposed below a surface of the object. Mounted on portion 19 is position transducer 22. Corresponding to position transducer 22 mounted on instrument 14 is position transducer 23 mounted on imaging transducer 21. Position transducer 22, mounted on instrument 14, may comprise a transmitter providing a positioning signal for reception by position transducer 23, which in this case would comprise a receiver. Additionally or alternatively, position transducer 23, mounted on imaging transducer 21, may comprise a transmitter providing a positioning signal for reception by position transducer 22, which in this case would comprise a receiver. Position transducers 22 and 23 are adapted to operate cooperatively to provide information regarding the position of instrument 14 relative to imaging transducer 21, as discussed in detail below.

For ease of discussion herein, it will be assumed that position transducer 22, mounted on instrument 14, comprises a transmitter and that position transducer 23, mounted on (or in) imaging transducer 21, comprises a receiver. However, it should be understood that the opposite could be true as well, and thus the claims should be interpreted with this understanding. The particular embodiment of the transmitter and receiver, their distribution between the instrument and imaging transducer, technique for their mounting, etc. depends upon factors such as size, shape, weight, cost, steribility, disposable or reusable parts. One example is to integrate the receiver with the imaging transducer (e.g., ultrasound probe) to facilitate the ease of use and simpler integration with the imaging system. Such an embodiment can readily be used as normal for imaging, with the receiver being available for interventional procedures. The receiver integrated into the imaging transducer may not be a disposable unit and/or its connections and power supply can be integrated into the cable for the imaging transducer. Another embodiment could treat the receiver as a clip-on or other removable appliqué to the imaging transducer or needle guide. Such a receiver may comprise a sterilable or disposable part. Data transfer to a corresponding processing unit (e.g., imaging system) for such an embodiment may be via wireless connection, using a battery pack. The corresponding transmitter plus its battery can be packaged together as a disposable unit which is a built-in or clipped-on part for the interventional instrument.

Position transducers 22 and 23 may be mounted on respective ones of instrument 14 and imaging transducer 21 using various techniques. For example, position transducer 22 may be mounted permanently to a sleeve or other cover into which instrument 14 is temporarily inserted, to thereby provide a reusable position transducer configuration where instrument 14 is itself disposable. Similarly, position transducer 23 may be permanently mounted on a bracket or sleeve which is removably attachable to imaging transducer 21. Alternatively, position transducers 22 and 23 may be permanently attached directly to a respective one of instrument 14 or imaging transducer 21. In some embodiments, position transducers 22 and 23 are adapted to be detachable, even from a sleeve, cover, or other bracket, to facilitate discarding or sterilization of this host structure. In this way the instruments and/or position transducer host structure can be discarded or sterilized independent from the position transducer. Because of sanitation and other housekeeping concerns (such as extra wires, calibration, etc.) it is anticipated that many embodiments will locate position transducer 23 within a housing of imaging transducer 21 and signals would be communicated with position transducer 22 associated with instrument 14 via a window or other signal transparent structure in the housing of imaging transducer 21.

Position transducer 22 may comprise a light transmitter, such as an active laser or light emitting diode (LED). Correspondingly, position transducer 23 may comprise a light detector or array of light detectors, such as may comprise a charge-coupled device (CCD) or photo diode. In the situation where a transmitter of the position transducers provides collimated light, a corresponding receiver of the position transducers can be, for example, a photo position sensitive detector (PSD) light detector, Embodiments of the invention may utilize position transducers in addition to or in the alternative to the aforementioned light transmitter and receiver, such as to use electrical, infrared, sound, magnetic, etc., transducer configurations for deriving a current position of the instrument according to the concepts herein. It should be appreciated that position transducer 22 mounted on instrument 14 can be battery powered, connected to a source of power by a conductor, comprise a photo-voltaic power source, etc. A receiver circuit of position transducer 32, such as may comprise a receiver, signal pre conditioner circuit, and analog-to-digital (ADC) converter may be provided with a wired or wireless interface with the imaging system.

In some embodiments, one of position transducers 22 or 23 may comprise a reflector or other passive element. In such an embodiment, the other one of position transducers 22 and 23 may correspondingly comprise both a transmitter and a receiver, operable to communicate via the reflector. Such configurations provide an implementation adapted to reduce the cost of a position transducer as disposed upon a particular component (e.g., instrument 14) to a point where the position transducer is easily disposable.

A position transducer pair (e.g., transmitter/receiver pair) of embodiments can be tuned to each other such that signals from other instruments are not acted upon. For example, such tuning can be provided by way of physical or electrical filters, lenses, polarizations, frequencies, amplitude or frequency modulation, etc.

FIG. 1b shows a superimposed overlay on an image generated using imaging transducer 21 in an out-of-plane technique (e.g., the configuration of FIG. 1a) according to an embodiment of the invention. Specifically, image 100 corresponds to image plane 16 and provides an image of features of the object beneath transducer surface 12 which would otherwise be invisible to the naked eye. The superimposed overlay provided with respect to image 100 shown in FIG. 1b includes predicted intersection pip 101 and instrument pip 102. Instrument pip 102 corresponds to the depth of tip 18 of instrument 14 and is used to show the depth of tip 18 as shown in reference frame 101′.

In the illustrated embodiment, the predicted intersection point of the instrument with the imaging plane is denoted by the “X” of predicted intersection pip 101 superimposed on the underlying image of image 100. Embodiments of the invention may provide a predicted intersection pip or other target designator appearing differently than illustrated in FIG. 1b, such as may have a distinctive color and/or shape denoting the desired target location. In operation, predicted intersection pip 101 may be superimposed to represent a predetermined distance below transducer surface 12, to correspond with a particular instrument guide configuration (e.g., angle of attack), may be positioned in accordance with clinician input provided to an imaging system unit, etc. For example, a clinician may dispose imaging transducer 21 to place a desired target in image plane 16, viewing image 100 in real-time to identify a particular target feature therein. Thereafter, the clinician may manipulate imaging transducer 21 and/or instrument guide 13 to position a desired target (e.g., a tumor, artery lumen, plaque, nerve, joint etc.) into predicted intersection pip 101. A processor of the imaging system unit may determine an appropriate instrument guide, or instrument guide setting (e.g., instrument guide angle), to provide guidance of instrument 14 for interfacing tip 18 with the target.

Instrument pip 102 is superimposed over the underlying image of image 100 and is preferably generated in real time (as will be discussed) to show a position of a portion of instrument 14, such as tip 18, relative to predicted intersection pip 101. For example, the position of instrument pip 102 may be based on physics (e.g., using instrument orientation data associated with the use of instrument guide 13) and the relative position of position transducers 22 and 23. Embodiments of the invention may provide an instrument pip or other instrument designator appearing differently than illustrated in FIG. 1b, such as may have a specific color and/or shape to make it easily distinguishable on image 100. Additionally or alternatively, embodiments of the invention may implement specific sounds or other sensory stimuli to indicate a position of the instrument relative to the target.

Line 103 (corresponding to the edge of reference frame 100′) shows an intersecting edge of the plane that instrument 14, guided by instrument guide 13, should be disposed in throughout its insertion into the object. Accordingly, movement of tip 18 should traverse line 103 longitudinally, as viewed in image 100, as instrument 14 is inserted into the object. Line 103 may be displayed as part of the superimposed overlay to aid a clinician or other user in envisioning the path of tip 18 according to embodiments. Alternative embodiments, however, may not display line 103 as part of the superimposed overlay.

As shown in FIG. 1b, instrument pip 102 is disposed above predicted intersection pip 101, which correlates to tip 18 being in front of image plane 16. That is, because instrument 14 has not yet been inserted deeply within the object, tip 18 is disposed more shallow within the object than the target and has not yet traversed image plane 16 in Which the target is disposed. It should be appreciated that, although an out-of-plane technique is being used, instrument pip 102 representing a relative position of tip 18 is shown on image 100 while tip 18 remains out of image plane 16. This can be seen more clearly in reference frame 100′ of FIG. 1b showing the relative depth position of tip 18, image plane 16, and predicted intersection pip 101. Specifically, reference frame 100′ shows a center cross plane of imaging plane 16 (image 100) that contains a portion of instrument 14 (e.g., the instrument shaft) and tip 18. As instrument 14 is inserted further into the object and tip 18 approaches image plane 16 along a diagonal in the instrument plane represented by line 103, image pip 102 will move down towards predicted intersection pip 101 on image 100.

Directing attention to FIG. 1c, the situation where instrument 14 has been inserted into the object sufficiently such that tip 18 has advanced to coincide with image plane 16 is shown. That is, instrument pip 102 corresponds to the depth of tip 18 of instrument 14 at a depth as shown in reference frame 101′ of FIG. 1c. This coincidence is represented in corresponding image 100 of FIG. 1d wherein predicted intersection pip 101 and instrument pip 102 are concentrically overlapping. In operation, a clinician monitors instrument pip 102 as instrument 14 is advancing through instrument guide 13 until instrument pip 102 is disposed in a predetermined relationship with predicted intersection pip 101. This predetermined relationship of instrument pip 102 and predicted intersection pip 101 indicates to the clinician that tip 18 is positioned directly on or in the target.

If instrument 14 is inserted further into the object than shown in FIG. 1c, tip 18 will traverse image plane 16 as shown in FIG. 1e. Correspondingly, instrument pip 102 will diverge below predicted intersection pip 101 on image 100 as shown in FIG. 1f. That is, instrument pip 102 corresponds to the depth of tip 18 of instrument 14 at a depth as shown in reference frame 101′ of FIG. 1e. Specifically, as instrument 14 is inserted further into the object and tip 18 passes image plane 16 along a diagonal in the instrument plane represented by line 103, image pip 102 will move deeper down into the object and away from image plane 16.

Embodiments of the invention operate to alert a clinician or other user of particular conditions with respect to the instrument and target. For example, embodiments may operate to change the color and/or shape of instrument pip 102 and/or predicted intersection pip 101 depending upon whether tip 18 is in front of, coincident with, or behind image plane 16. Additionally or alternatively, flashing, flashing frequency, tones or other sounds, size, color, or shape of the pip may be provided to indicate the relative proximity of tip 18 to the target. For example, a green pip may indicate that the tip has not intersected the imaging plane, a white pip may indicate that the tip is intersecting the imaging plane, and a red pip may indicate that the tip has proceeded past intersecting the imaging plane.

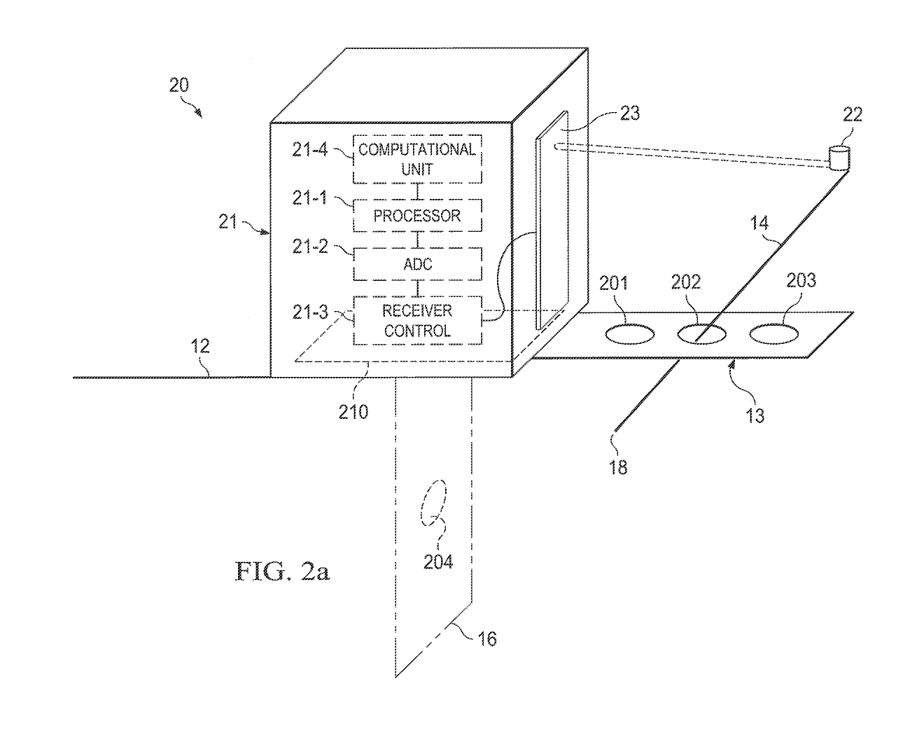

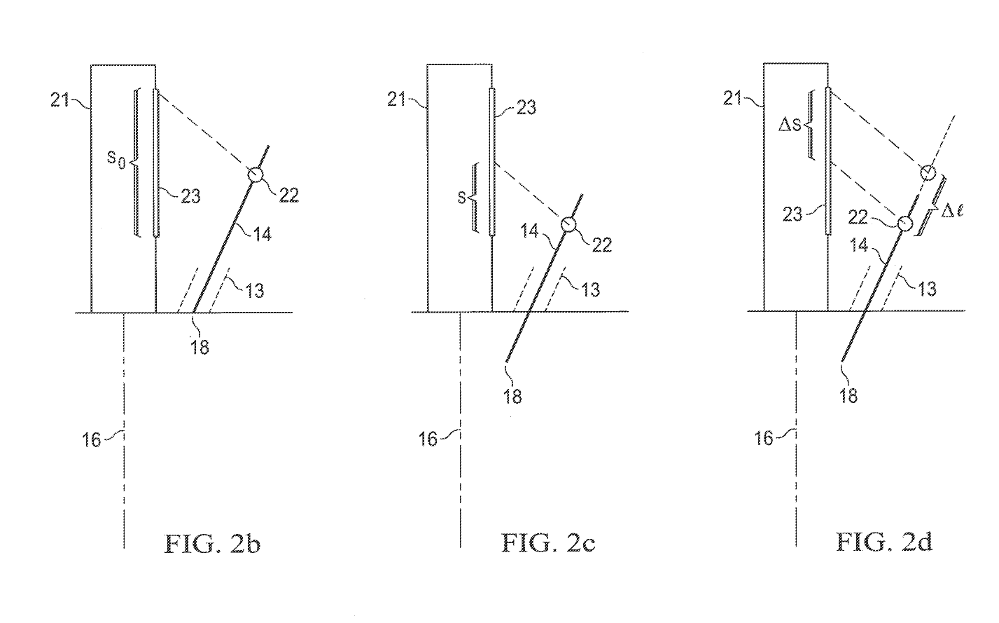

FIG. 2a shows a schematic view of embodiments of the present invention to illustrate operational principals of the concepts herein. It should be appreciated that although the illustrated embodiment shows only imaging transducer 21 of imaging system 20, imaging system 20 may comprise additional components. For example, embodiments of the invention include a system unit providing signal amplification, control, analog-to-digital conversion, signal processing, image generation, and other functions in cooperation with imaging transducer 21. Several of the functional blocks may be disposed in such a system unit and/or imaging transducer 21, as desired. For example, any or all of processor 21-1, ADC 21-2, receiver control 21-3, and computational unit (e.g., ARM, CPU, DSP, FPGA, SOC., etc.) 21-4 shown disposed in imaging transducer 21 may be disposed in an associated system unit (not shown) of imaging system 20, if desired.

Transducer 210 is shown in imaging transducer 21 to illustrate that position transducer 23 of embodiments comprises transducer apparatus apart from transducer 210 typically used in generating an image with imaging transducer 21. Although the particulars of transducer 210 are not critical to implementation of the concepts herein, a general description of an exemplary transducer configuration is provided for completeness. Transducer 210 may, for example, comprise an array of ultrasound transducers operable to transmit Ultrasonic pulses into an object and receive reflected and/or generated harmonic ultrasonic signals therefrom, These received ultrasonic signals may be processed by processor 21-1 or another processor (not shown) for generating a sonographic image (e.g., the underlying image of image 100).

As shown in FIG. 2a, instrument 14 is interfaced with instrument guide 13 to provide control of instrument 14 as the instrument is inserted into an object. Instrument guide 13 is shown with different angle of attack guides 201, 202, and 203 for guiding instrument 14 to different depths below surface 12. In the illustrated embodiment, the target (e.g., a tumor, artery lumen, plaque, nerve, joint etc.) is depicted as target 204 disposed below surface 12, and is thus invisible to a clinician or other operator of imaging system 20. Nevertheless, an appropriate one of angle of attack guides 201-203 will facilitate insertion of instrument 14 to interface with target 204. However, without operation of a superimposed overlay of embodiments of the present invention, a clinician or other user of imaging system 20 may not accurately determine when tip 18 interfaces with target 204.

According to an exemplary embodiment of the system in FIG. 2a, position transducer 22 comprises a laser source. Light from the laser source of position transducer 22 preferably illuminates portions of a PSD receiver of position transducer 23 as instrument 14 is guided by instrument guide 13. Preferred embodiments implement at least dual-channel communication and circuitry to filter out ambient light or other interferences with respect to a PSD receiver of position transducer 23. Embodiments may additionally or alternatively implement circuitry to amplify the signal, provide analog-to-digital conversion, provide signal processing, computation to derive the tip location, etc,

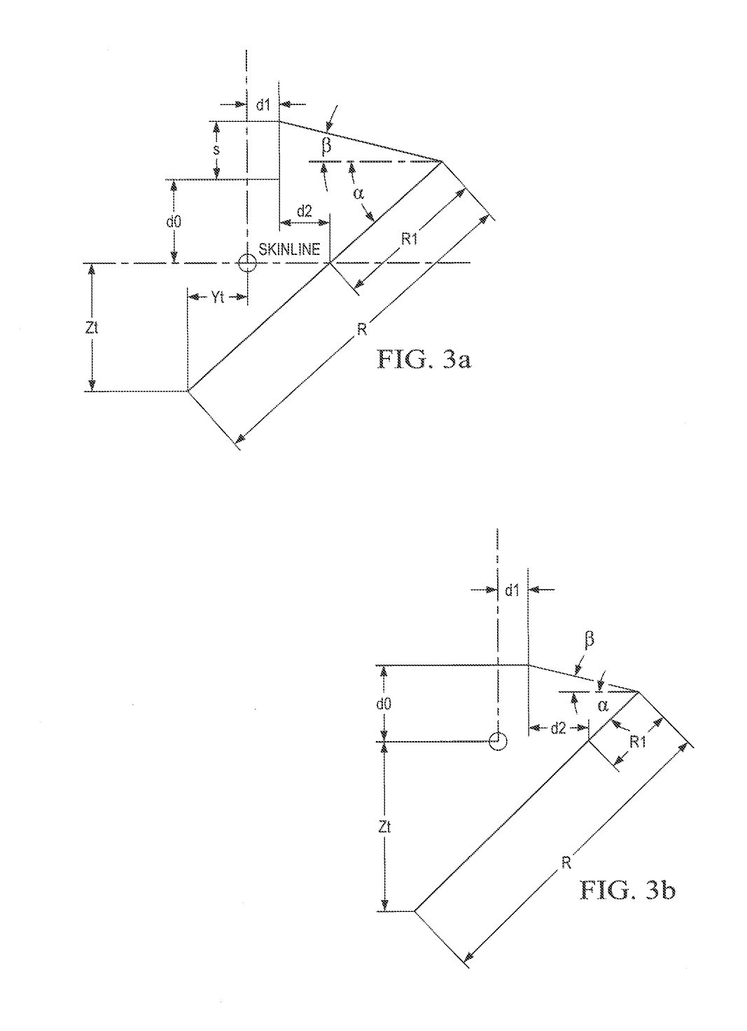

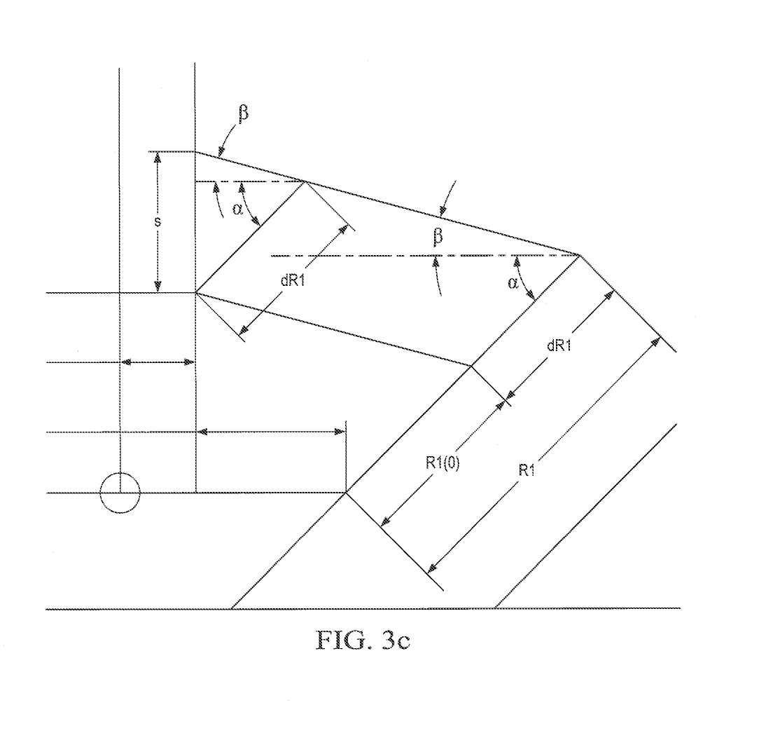

In operation, the location of instrument 14, or a portion thereof (e.g., tip 18) is calculated using position information obtained using position transducers 22 and 23. For example, processor 21-4, operating from information received via receiver control 21-3 and (if necessary) ADC 21-2, may calculate a position of tip 18 as discussed in detail with respect to FIG. 3 below. It should be appreciated that the calculations, or portions thereof, may be made external to imaging transducer 21, such as by transmitting information to a remote processor (e.g., the aforementioned system unit). As will be discussed, the processor would contain one or more applications (or firmware) to perform the geometric calculations necessary to estimate the exact position of the tip (or other portion of the instrument) and to then generate the proper display for superimposing the calculated position of the tip over the actual sonographic image.

FIGS. 2b-2c illustrate operation of the embodiment of FIG. 2a to provide location determinations for instrument 14. In FIG. 2b, an initial state of instrument 14 is used for calibration, and for setting the starting coordinates for tip 18 of instrument 14 (as discussed in further detail below). In FIG. 2c, instrument 14 is advanced along the path defined by instrument guide 13. The relationship between the linear distance difference As on the sensor, and the corresponding linear distance difference Al along the path of instrument 14 is shown (as discussed in further detail below).

A plurality of methods can be used to determine the geometric relationship between a transmitter and receiver utilized according to embodiments of the invention. One such method to determine the geometric relationship between a transmitter and receiver comprises a fixed location configuration, whereas another such method comprises calibrating the geometric relationship prior to use. The mathematical bases for each of the foregoing methods is provided below.

A fixed location configuration of embodiments utilizes a predetermined, fixed location of the transmitter on an instrument. For example, the fixed position can be a predetermined mounting position for the user to attach the transmitter, the mounting may be performed in the factory, etc. The geometric relationship of the transmitter and receiver may thus be predetermined. Accordingly, with a fixed location of the transmitter on an instrument, no user calibration is necessary according to embodiments of the invention.

A calibration routine may be executed prior to beginning a procedure using a superimposed overlay of embodiments of the invention. A calibration technique as may be utilized according to embodiments of the invention places one or more markers on the instrument, where such markers are at fixed location(s) from a portion of interest of the instrument (e.g., the tip). By placing a position transducer at a known location, as designated by the foregoing markers, calibration of the position transducer and instrument end, or other feature, can be established based upon the marker position. Such an embodiment avoids using an artificially created surface plane of the previously described embodiment.

It should be appreciated that particular situations may suggest that one or the other such methods should be utilized. For example, the fixed location configuration may limit the type of instruments being used, However, the calibration configuration may require an extra step for the user to perform the calibration.

FIG. 3a shows geometric coordinate system of the basis for calculating instrument positioning according to embodiments of the invention. It should be appreciated that the view provided in FIG. 3a is in-plane with respect to the plane that instrument 14, guided by instrument guide 13, should be disposed in throughout its insertion into the object and is out with respect to image plane 16, Accordingly, the line shown by the Z axis in FIG. 3a represents an edge of image plane 16 according to embodiments.

In the geometric construction of FIGS. 3a-3c, the goal is to determine the coordinate (Yt, Zt) of the instrument tip. The parameters used in FIGS. 3a-3c are:
s=position measurement along sensor, from its lower edge
d₀, d₁=fixed dimensions in the mechanism
d₂=fixed dimension from sensor plane to needle penetration point.
α=needle angle (from horizontal)
β=laser beam angle (from horizontal)
R=overall needle length
R₁=length of needle above skin line
   The values of d₀, d₁, d₂, R, α, β are known from the imaging transducer and instrument guide configurations and may be stored for use in a database (e.g., a database of computational unit 21-4 of FIG. 2a) according to embodiments of the invention.

For the case where s=0, R₁₍₀₎ can be found from the simplified diagram of FIG. 1b. As can be derived from the geometry of FIG. 1b, d₀=R₁₍₀₎sin α+(R₁₍₀₎ cosα+d2)tan β or d₀−d₀−d₂ tan β=R₁₍₀₎(sin α+cos α tan β). Thus:

$${R}_{1 ( 0 )} = \frac{{d}_{0} - {d}_{2} tan β}{sin α + cos α tan β} \qquad ( 1 )$$

As R₁ is increased from R₁₍₀₎ by dR₁, the laser strike point position s can be found from the diagram of FIG. 3c. The triangle on the upper left can be constructed from simple geometry. The position measurement along the sensor, s, may be represented as s=dR₁ sin α+dR₁ cos α tan β or s=dR₁(sin α+cos α tan β). Rearranging provides:

$${dR}_{1} = \frac{s}{( sin α + cos α tan β )} \qquad ( 2 )$$

Since R₁=R₁₍₀₎+dR₁, equations (1) and (2) may be used to provide

$${R}_{1} = \frac{{d}_{0} - {d}_{2} tan β}{sin α + cos α tan β} + \frac{s}{( sin α + cos α tan β )} ,$$

which simplifies to:

$${R}_{1} = \frac{{d}_{0} - {d}_{2} tan β + s}{sin α + cos α tan β} \qquad ( 3 )$$

From FIGS. 3a and 3b, it can be seen that

$$sin α = \frac{{Z}_{1}}{( R - {R}_{1} )} ,$$

so Z₁=(R−R₁)sin α. Substituting equation (3) gives Zₜ as:

$${Z}_{t} = [ R - \frac{{d}_{0} - {d}_{2} tan β + s}{sin α + cos α tan β} ] sin α \qquad ( 4 )$$

Having determined Zₜ, Yₜ can he determined from:

Yₜ=Zₜ*cot α−(d₁+d2) (5)

For the aforementioned in-plane method, both Zₜ and Yₜ, and the scale factor for the image are utilized to generate the tip location on the imaging plane according to embodiments. For the aforementioned out-of-plane method, Zₜ and the scale factor for the image are utilized to generate the tip location on the imaging plane according to embodiments.

As previously mentioned, it may be desirable to provide a calibration routine, such as may be executed prior to beginning a procedure using a superimposed overlay of embodiments of the invention. In a calibration routine implemented according to embodiments of the invention, a known surface plane is established and the instrument is advanced to touch the surface plane. When intersection occurs, the system knows the exact location of and end of the instrument (e.g., an instrument tip) as well as the location of the position transducer which moves with the instrument. From this information further movement of the instrument (after removing the artificially created surface plane) causes movement of between the corresponding position transducers and the location of the instrument, or its end, can then be precisely estimated for superimposing on a generated image, or for other purposes. In the foregoing exemplary embodiment, an objective of the calibration is to find the fixed geometric relationship between the position transducer and the instrument end.

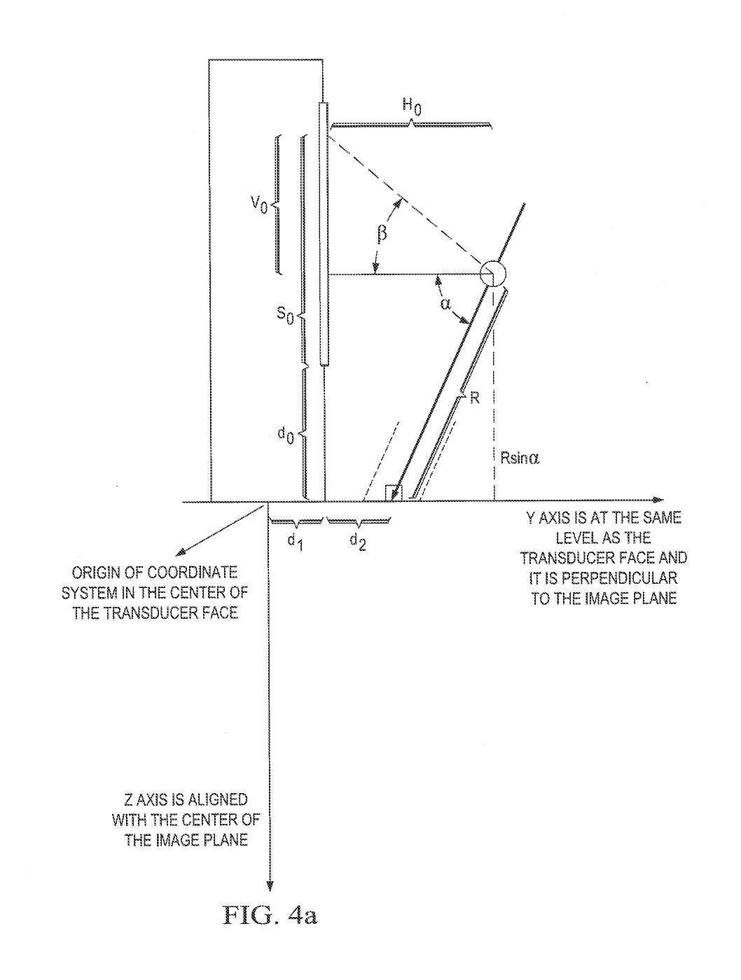

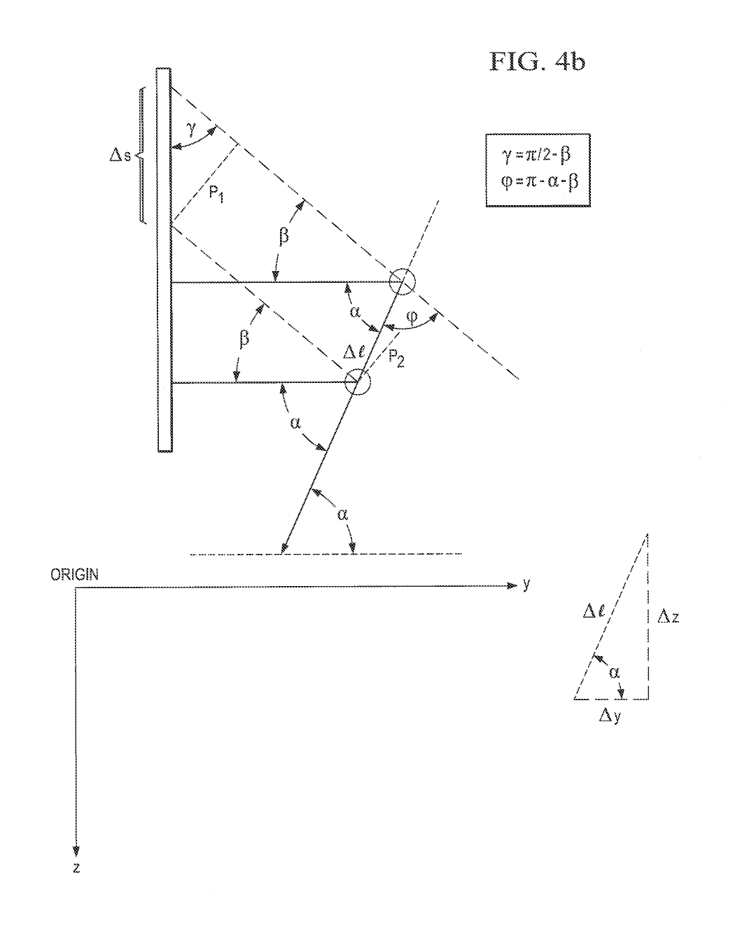

FIGS. 4a and 4b and the equations below illustrate a calibration procedure and use of an optical sensor for computation of the instrument tip coordinates with respect to an image plane according to an embodiment. The calibration procedure as illustrated in FIG. 4a is used to compute angle β, and if desired the distance R between a position transducer (e.g., light source) disposed upon the instrument and the tip of the instrument. This information may be utilized to compute the instrument tip coordinates as illustrated in FIG. 4b.

The calibration procedure of embodiments comprises inserting an instrument in an instrument guide (e.g., a fixed-angle needle guide). A position transducer, such as a light source (e.g., laser beam), is mounted on the instrument, A fixture (not shown) is attached to the imaging transducer such that it can be used for ensuring that the tip of the instrument is in the same z-level as the imaging transducer face. FIG. 4a shows the defined coordinate system and the geometry details of the foregoing calibration configuration.

By observing the triangle containing angle β with sides H₀ and V₀ the following can be derived:

$$tan β = \frac{{V}_{0}}{{H}_{0}} = \frac{{s}_{0} + {d}_{0} - R \cdot sin α}{{d}_{2} + R \cdot cos α} ⇒ β = arctan ( \frac{{d}_{0} - R \cdot sin α + {s}_{0}}{{d}_{2} + R \cdot cos α} ) \qquad ( 6 )$$

Using equation (6) the angle β of a light emitted from a position transducer disposed on the instrument (e.g., a laser beam) may be calculated based on the following variables:
distances d₀ and d₂ (e.g., as may be known based on the mechanical design);
angle α (e.g., as may be known based on the needle guide mechanical design);
length R as may be known or as may be computed using a calibration step); and
distance s₀

The distance s₀ along the position transducer (e.g., light sensor) can be computed by the currents received from the position transducer and its characteristic equation. The characteristic equation for a light sensor, as may sense a light beam emitted by a corresponding light source disposed upon the instrument, is as follows:

$${s}_{0} = \frac{L}{2} \cdot ( 1 - \frac{{i}_{2} - {i}_{2}}{{i}_{1} + {i}_{2}} ) \qquad ( 7 )$$

In the foregoing, L is the length of the sensor.

Furthermore, the configuration shown in FIG. 4a may be used to associate a sensor distance s₀ with corresponding values for the initial instrument tip coordinates y and z (denoted as y₀ and z₀).

Based on the way the fixture is specified and the way the coordinate system is defined it may be observed that:

y₀=d₁+d₂ (8)

z₀=0 (9)

The relationship between a linear distance difference at the sensor and the corresponding linear distance difference along the path of the instrument (i.e., the relationship of Δs to Δl) may be determined from the geometrical relationships illustrated in FIG. 4b, Specifically, FIG. 4b shows how the linear distance differences in a sensor can be translated to linear differences along the instrument path.

Observing the triangle containing segment Δs and angle γ in FIG. 4b, it can be seen that side P₁ of this triangle is drawn such that it is vertical to a light beam between the position transducers.

$${P}_{1} = Δ s \cdot sin γ = Δ s \cdot sin ( \frac{π}{2} - β ) = Δ s \cdot cos β \qquad ( 10 )$$

Observing the triangle containing segment Δl and angle φ in FIG. 4b, it can be seen that side P₂ of this triangle is drawn such that it is vertical to a light beam between the position transducers.

P₂=Δl·sin φ=Δl·sin(π−α−β)=Δl·sin(α+β) (11)

As can be appreciated from the illustration of FIG. 4b, P₁=P₂. Thus:

$$Δ s \cdot cos β = Δ 𝓁 \cdot sin ( α + β ) ⇒ Δ 𝓁 = Δ s \cdot \frac{cos β}{sin ( α + β )} \qquad ( 12 )$$

Using angle a and the triangle shown in FIG. 4b, a distance along the instrument path, can be computed. In particular, the following relationships may be derived. from the configuration shown in FIG. 4b:

$$Δ γ = Δ𝓁 \cdot cos α = - Δ s \cdot \frac{cos α \cdot cos β}{sin ( α + β )} \qquad ( 13 )$$

The minus sign of Δs in equation (13) indicates that as s becomes larger (e.g., light moves down the sensor in the positive z direction) y becomes smaller.

$$Δ z = Δ𝓁 \cdot sin α = Δ s \cdot \frac{sin α \cdot cos β}{sin ( α + β )} \qquad ( 14 )$$

The plus sign of Δs in equation (14) indicates that as s becomes larger (e.g., light moves down the sensor in the positive z direction) z becomes larger.

By combining equation (13) with equation (14) the equation that describes the y coordinate of the instrument tip as the user moves the instrument may be determined:

$$y = {y}_{0} - Δ s \cdot \frac{cos α \cdot cos β}{sin ( α + β )} ⇒ y = {d}_{1} + {d}_{2} - Δ s \cdot \frac{cos α \cdot cos β}{sin ( α + β )} \qquad ( 15 )$$

Similarly the equation that describes the z coordinate of the instrument tip as the user moves the instrument may be determined:

$$z = {z}_{0} + Δ s \cdot \frac{sin α \cdot cos β}{sin ( α + β )} ⇒ z = Δ s \cdot \frac{sin α \cdot cos β}{sin ( α + β )} \qquad ( 16 )$$

Using equations (15) and (16) visual feedback may be provided to the user about the coordinates of the instrument tip, such as in the form of instrument pip 102 (FIGS. 1b, 1d, and 1f) superimposed upon a generated image. Additionally or alternatively, information such as the instrument tip distance (e.g., in mm) from the image plane along the y axis and/or from the imaging transducer face along the z axis may be provided.

The foregoing operation is summarized in the following step-wise procedure:
1. Insert the instrument in the instrument guide and advance the instrument such that the instrument tip is at the same z-level as the imaging transducer face. A mechanical fixture may be utilized to ensure that the instrument tip is at the same z-level as the imaging transducer face.
2. Record sensor measurement.
   a. Compute s₀ (distance from bottom of sensor to point at which optical sensor light beam strikes sensor) by using equation (7).
   b. Store s₀ for future use.
   c. Compute angle β from equation (6).
   d. Computer y₀ and z₀ using equations (8) and (9) respectively.
   e. Store y₀ and z₀ for future use.
3. Remove the fixture (if used in the calibration process) and advance instrument to perform desired procedure.
   a. Compute new s (distance from bottom of sensor to point at which optical sensor light beam strikes sensor) by using equation (7).
   b. Compute Δs (Δs=s−s₀).
   c. Compute the updated y and z coordinates using equations (15) and (16)

Using the geometric formulations discussed above, processor 21-1 of embodiments determines the relative location within image 100 of one or more portion of instrument 14, such as tip 18. For example, calculation of the depth z provides information regarding where tip 18 is disposed on line 103 (FIGS. 1b, 1d, and 1f). Thus processor 21-1 may create (or provide information, to another processor, such as an image processor of an associated system unit, not shown) a graphic display (e.g., pip) representing the disposition of tip 18 (or any other desired portion of instrument 14), such as instrument pip 102, for use as a superimposed overlay on an underlying image.

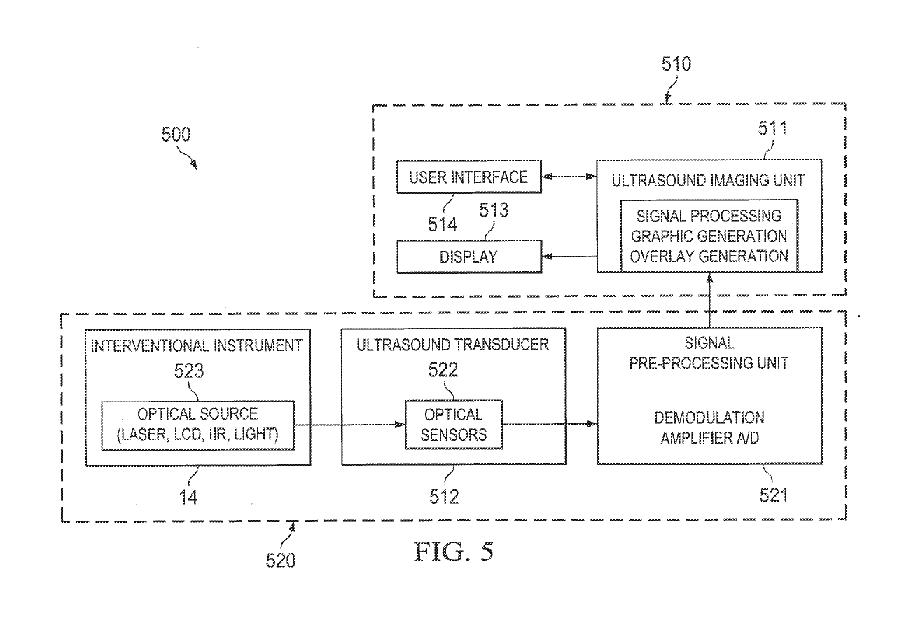

FIG. 5 shows detail with respect to the distribution of functional blocks of an imaging system adapted according to embodiments of the invention. Imaging system 500 of the illustrated embodiment comprises imaging system unit 510 having imaging unit 511, imaging transducer 512, display 513, and user interface 514. Optical sensor system 520 of the illustrated embodiment includes signal processing unit 521, optical sensor 522, and optical source 523. Signal processing unit 521 of the illustrated embodiment provides such signal processing functions as demodulation, amplification, analog-to-digital and/or digital-to-analog conversion, etc. Imaging unit 511 of the illustrated embodiment provides such imaging functions as signal processing, graphic generation, overlay generation, etc. It should be appreciated that the signal pre-processing and signal processing to derive the tip spatial location can all be done outside the imaging unit, if desired. However, the illustrated example shows such functions to be provided in the imaging unit to make use of existing computational and graphic capability. Display 513 of embodiments provides display of a generated image and superimposed position graphics. User interface 514 of embodiments allows the user to control (e.g., turn on/off, select operating parameters, etc.) the imaging system and turn the instrument position determination feature.

It should be appreciated that, although the foregoing example is provided with respect to an instrument which is linear and having a fixed length between the position transducer on the instrument and the instrument portion of interest (e.g., the tip), the concepts of the present invention are applicable to different instrument configurations. In particular, the concepts discussed herein may he utilized with compounded shapes and/or variable lengths. For example, with a curved instrument (e.g., curved needle) the calculations would include the curve dimensions and would project where the end would be even though it was not a straight line calculation. For variable length instruments, embodiments would be provided with, or calculate, the length (distance from the position transducer to a given point on the instrument) at any given time. One technique for knowing the length at any give time is to mark the instrument at intervals (or with codes) and use these interval markers, or codes, to know the length of the instrument at any point in time. Such markers could be used to determine the instantaneous R dimension (FIGS. 3a-3c) and the tip or other portion of instrument can be calculated knowing this instantaneous R dimension.

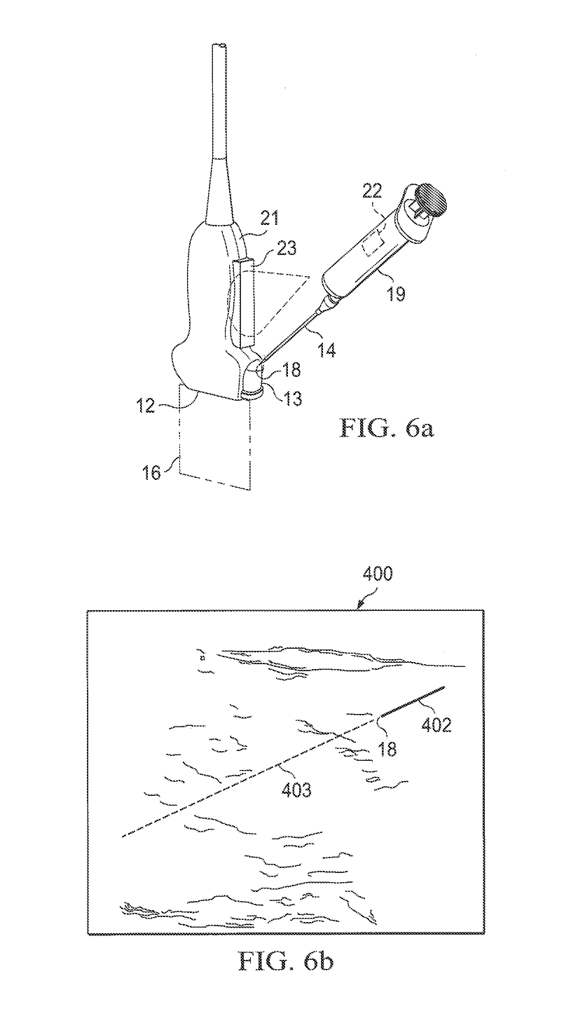

Although embodiments have been described with reference to an out-of-plane technique, it should be appreciated that the foregoing concepts are applicable to in-plane techniques. Accordingly, FIG. 6a shows an illustration of an embodiment of the invention adapted to facilitate positioning an instrument using an in-plane technique. In the embodiment of FIG. 6a, position transducer 23 mounted on imaging transducer 21 has been moved (as compared to the out-of-plane embodiment of FIG. 1a) from the front of the imaging transducer to the side of the imaging transducer. Correspondingly, instrument guide 13 has been moved (again, as compared to the out-of-plane embodiment of FIG. 1a) from the front of the imaging transducer to the side of the imaging transducer. Nevertheless, position transducer 23 continues to work in cooperation with position transducer 22 mounted on instrument 14 according to the concepts discussed above as instrument 14 is guided into the object disposed below imaging transducer 21. However, because instrument 14 is being inserted into the object in the same plane as image plane 16 (as controlled by instrument guide 13) the resulting image provides a long axis view of instrument 14, whereby a longitudinal portion of instrument 14 may be visualized. The instrument guide keeps the instrument in the imaging plane at a fixed angle.

FIG. 6b shows a superimposed overlay on an image generated using imaging transducer 21 in an in-plane technique (e.g., the configuration of FIG. 6a) according to an embodiment of the invention. Specifically, image 400 corresponds to image plane 16 and provides an image of features of the object beneath surface 12 which would otherwise be invisible to the naked eye. The superimposed overlay provided with respect to image 400 shown in FIG. 6b includes projected trajectory 403 representing a path along which instrument 14 is projected to follow, as may be determined by a particular instrument guide selected, an angle of attack used, etc. Embodiments may provide a plurality of such projected lines, such as corresponding to various settings or angles of attack available using instrument guide 13. Also included in the superimposed overly of FIG. 6b is graphical instrument designator 402 corresponds to a portion of instrument 14 inserted into the object and used to show the position of instrument 14 relative to a desired target. It should be appreciated that graphical instrument designator 402 of the illustrated embodiment provides a clear representation of the end of instrument 14, and thus provides position information regarding tip 18 within the object.

As discussed above, the graphical objects of the superimposed overlay (e.g., graphical instrument designator 402 and projected line 403) can have a particular shape, color, etc. as desired. For example, although the foregoing in-plane technique lends itself to providing a longitudinal representation of instrument 14 as shown by the illustrated embodiment of graphical instrument designator 402, embodiments may utilize different shaped designator such as an instrument pip described above.

In operation, a clinician may manipulate imaging transducer 21 so that projected line 403 passes through a desired target (e.g., a tumor, artery lumen, plaque, nerve, joint etc.). Thereafter, the clinician may insert instrument 14 into or near the region of interest, guided by instrument guide 13. Because instrument 14 will progress along a longitudinal axis of image plane 16 (e.g., the instrument is inserted in-plane), the instrument can be represented by graphical instrument designator 402, preferably in real-time, to show instrument 14 progressing along projected line 403. The position of instrument 14 within the object, and thus the position of graphical instrument designator 402, may be determined using the techniques discussed above with respect to FIGS. 3a-3c, The clinician may cease further insertion of instrument 14 when graphical instrument designator 402 is viewed to interface with a desired target appearing in image 400. This is particularly useful for steep angle insertion when the image of the instrument is poor or not visible at all due to specula reflection.

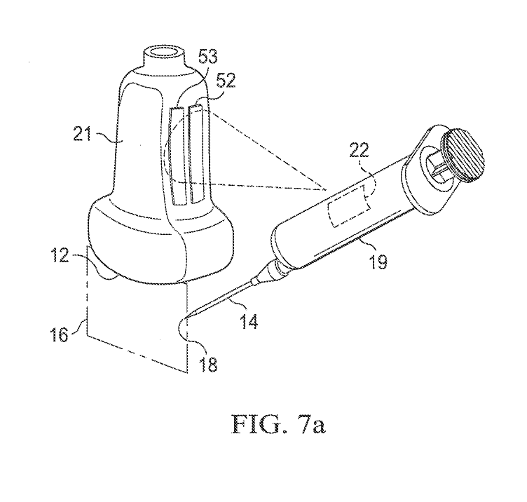

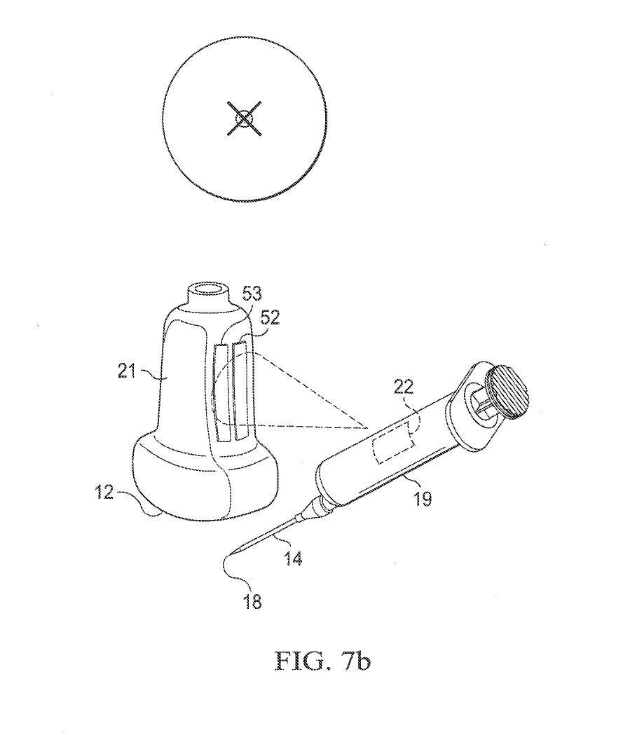

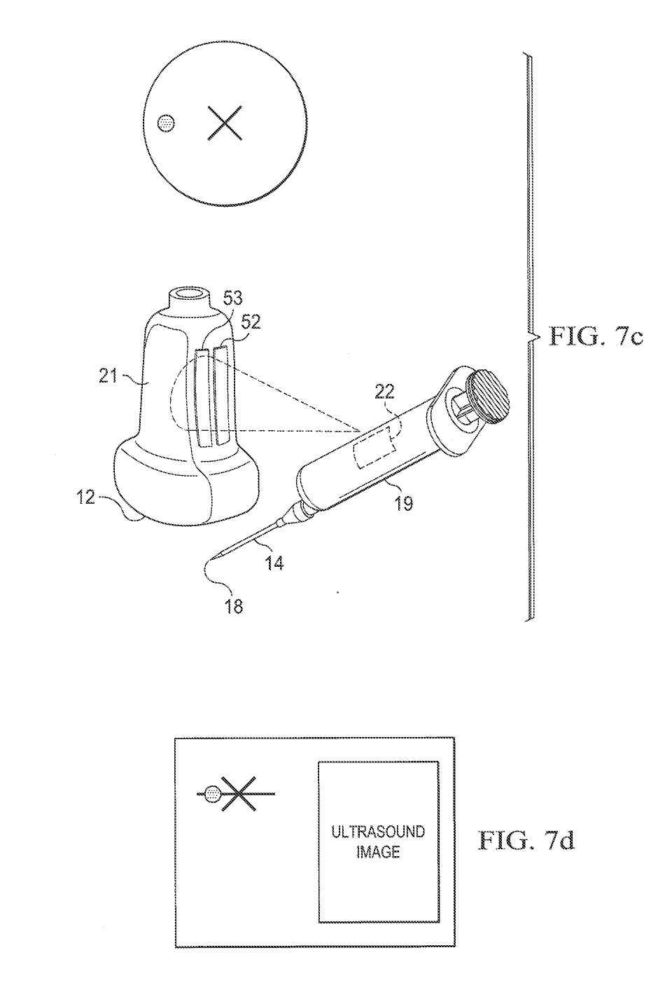

FIG. 7a shows an embodiment of an optical sensor system of the present invention. In a use case of the embodiment of FIG. 7a, the optical sensor system may be utilized for detecting if the instrument is located within the imaging plane in addition to or in the alternative to operating to locate the instrument or a portion thereof.

In operation according to an embodiment of the configuration shown in FIG. 7a, a plurality of position transducers, Shown here as position transducers 52 and 53 (e.g., optical receivers or a PSD device), are used to deduce (e.g., triangulate) the position of a plane that contains instrument 14 relative to imaging plane 16. When instrument 14 is inside the imaging plane, the signals from position transducers 52 and 53 resulting from illumination by position transducer 22 (or the two outputs i1 and i2 from a PSD device) will be equal according to an embodiment. Thus an indication that instrument 14 is in imaging plane 16 may be provided to a user, as represented by the coincidence of the pips in FIG. 7b. If, however, instrument 14 is not inside the imaging plane, the signals from position transducers 52 and 53 resulting from illumination by position transducer 22 (or the two outputs i1 and i2 from a PSD device) will not be equal according to an embodiment of the invention. Thus an indication that instrument 14 is out of imaging plane 16 may be provided to a user, as represented by the separation of the pips in FIG. 7c.

The following equation gives the position of the plane the instrument is disposed in relative to the imaging plane:

$$Y = \frac{L}{2} * ( \frac{i 1 - i 2}{i 1 - i 2} ) \qquad ( 17 )$$

In the foregoing equation:
Y is the instrument plane offset from the imaging plane;
L is the length of the PSD device; and
i1 and i2 are the current out from the position transducers or PSD.

FIG. 7d shows a sample graphic display which may be presented to a user according to embodiments of the invention to provide information regarding the plane of the instrument relative to the imaging plane. In particular, FIG. 7d shows a reference graphic display that can be located near or on the generated image. The reference graphic of the illustrated embodiment contains the imaging plane location donated by X and the instrument plane donated by a small dot, In real-time, the dot is moving according to the hand movement guiding the instrument. The user would observe the movement of the dot and try to move it to where the X is and maintain it there. This method allows the user to concentrate on the monitor display where the generated image is displayed without looking down or to the side to see where their hand is. it gives the user both the generated image and instrument plane information in a single scan of the user's vision. This visual aid can reduce hand-eye coordination issues.

Concepts of the present invention have been described herein with reference to particular illustrated embodiments. However, it should be appreciated that embodiments may deviate significantly from those of the illustrated embodiments and yet the concepts herein may be utilized to facilitate the correct placement of an instrument internal to an object aided by an overlay superimposed on an image. For example, a position transducer need not be mounted on the imaging transducer according to embodiments, so long as the relationship between the imaging transducer and the instrument can be determined. Similarly, it is expected that other technologies may be employed to determine the geometric relationships between an instrument and an imaging plane in order to perform calculations necessary to overlay a calculated position of a portion of the instrument onto an image without use of a needle guide or, in the case of a “free-hand” insertion, a plurality of transducers. According to some embodiments, an instrument guide (e.g., instrument guide 13) may have position transducer 23 and/or other sensor apparatus mounted thereto or otherwise associated therewith. For example, the instrument guide can be adapted such that the current angle of attack being utilized is determined by a sensor and presented to the processor for use in calculating the anticipated position of the instrument.

Although embodiments have been described herein with reference to ultrasound imaging systems, it should be appreciated that the concepts of the present invention. are applicable to a number of technologies. For example, embodiments of the present invention may be provided with respect to other image generation devices, such as fluoroscope systems, tomography systems, etc.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of indicating a position of an instrument inserted in an object on an image generated using an imaging transducer, said method comprising:
establishing optical communication between at least one point on said instrument and at least one point on said imaging transducer;
moving said instrument relative to said imaging transducer;
calculating positions of at least a portion of said instrument relative to Said generated image, said calculating dependant at least in part on relative positioning between said points as determined through said optical communication; and
indicating a current position of said at least a portion of said instrument in said generated. image by superimposing a graphical instrument designator overlay on an underlying image generated using said imaging transducer.

2. The method of claim I further comprising:
designating a predicted image plane intersection point for said instrument;
indicating a position of said predicted image plane intersection point in said generated image by superimposing a graphical predicted intersection designator overly on said underlying image.

3. The method of claim 2 further comprising:
manipulating said imaging transducer to dispose said graphical predicted intersection designator coincident with a target within said underlying image.

4. The method of claim 1 further comprising:
changing at least one of a shape, a size, a color, and a sound as said graphical instrument designator is moved relative to said graphical predicted intersection designator.

5. The method of claim 1 further comprising:
superimposing a predicted trajectory of said instrument through said object on said underlying image.

6. The method of claim 1 wherein said calculating comprises:
establishing a known and fixed angle of attack between said instrument and said imaging transducer; and
processing geometric calculations based on said angle of attack and distances associated with said points.

7. A method of indicating a position within an object of an instrument used in conjunction with an imaging system, said method comprising:
tracking movement between a known position on said imaging system and a position on said instrument, said position on said instrument being a known distance from a particular portion of said instrument inserted into said object; and
calculating a position of said particular portion of said instrument, said calculating dependant at least in part on said tracking movement between said known positions using optical communication; and
indicating a current position of said particular portion of said instrument in an image by superimposing a graphical instrument designator overlay on an underlying image generated by said imaging system.

8. The method of claim 7 wherein said tracking comprises:
passing laser light in at least one direction between said known positions.

9. The method of claim 7 further comprising:
indicating a position of a target within said object by superimposing a graphical predicted intersection designator overlay on said underlying image, wherein said graphical instrument designator and said graphical predicted intersection designator show a relative position of said particular portion of said instrument and said target.

10. The method of claim 9 wherein said calculating comprises:
establishing an angle of attack between said instrument and said generated image; and
processing geometric calculations based on said angle of attack and known distances associated with said known positions.
